(19) United States
(12) Patent Application Publication
(10) Pub. No.: US 2015/0289984 A1
(43) Pub. Date: Oct. 15, 2015

(54) Total Shoulder Arthroplasty Prosthesis

(71) Applicant: Matthew D. BUDGE, Tualatin, OR (US)

(72) Inventor: Matthew D. BUDGE, Tualatin, OR (US)

(21) Appl. No.: 14/253,751

(22) Filed: Apr. 15, 2014

(51) Int. Cl. A61F 2/40

(52) U.S. Cl. CPC A61F 2/40; A61F 2/4081; A61F 2/4059; A61F 2002/4085

(57) ABSTRACT

A glenohumeral arthroplasty prosthesis with a biocompatible metal glenoid component and a biocompatible metal humeral component with a single-fixation adjustable head having a crosslinked ultra high molecular weight polyethylene articular surface thereupon.

### CONTINUITY AND CLAIM OF PRIORITY

This is an original U.S. patent application that claims priority to U.S. provisional patent application No. 61/949,203 filed 6 Mar. 2014.

### FIELD

The invention relates to prostheses used in shoulder arthroplasty. More specifically, the invention relates to implantable prostheses for use in shoulder joint reconstruction surgery.

### BACKGROUND

The shoulder is one of the most complex and mobile joints in the human body. However, it is also susceptible various types of injury and arthritis which can cause pain and dysfunction via the loss of the smooth cartilage lining the surfaces of the joint.

One of the main treatments for shoulder pain resulting from arthritis or injury is Total Shoulder Arthroplasty (TSA). In this procedure arthritic bone is removed from the ball-and-socket joint and a shallow plastic (polyethylene) cup is placed in the glenoid (socket). A metal hemisphere and stem combination are subsequently used to replace the humeral head (ball) of the ball-and-socket-joint. Replacing the arthritic bone with metal and plastic surfaces allows for pain relief and restoration of normal function of the shoulder.

The “ball” half of a total shoulder arthroplasty is routinely a stem made of a biocompatible metal which is implanted within the bone of the humerus. A metal hemisphere is then attached to the most proximal end of the stem to reproduce the curved articulation with the socket. This portion of the prosthesis can be implanted with bone cement (polymethylmethacrylate) or by placing the implant tightly within the bone to encourage the bone to grow into the metal of the stem.

The “socket” half of the replacement is most commonly an all-polyethylene cup which is secured to the glenoid by drilling/compacting a hole into the bone, placing bone cement within the holes, and then impacting the polyethylene glenoid component. The bone cement acts as a grout to secure the polyethylene implant to the bone.

The primary limitation of the previously mentioned glenoid design is that there is no biologic ingrowth of bone into the polyethylene or the cement which would lead to permanent long term stability of the implant. As a result, the life span of the glenoid half of a total shoulder arthroplasty is only 10-15 years. This is a significant limitation which restricts the usefulness of glenoid components in young patients who would require multiple revisions, and has led to a growing number of revision surgeries for failed glenoid implants which can have poor outcomes.

One of the main reasons for the short longevity of the glenoid half of a total shoulder replacement is the limited bone available in the glenoid fossa for implantation of a prosthetic component. The socket part of the ball-and-socket is shallow and has a surface area of only 4-10 cm² (by comparison the hip, the largest ball-and-socket joint in the body, has a socket with a surface area of around 30 cm²). The size relationship between the humeral head (ball) and glenoid (socket) has been referred to as a “golf ball on a tee.” Since the area of bone for glenoid implantation is small, the implant itself must be smaller still to fit comfortably on the bone. However, the implant must also be durable enough to resist the repetitive forces of the shoulder joint without early loosening and failure. The correct balance between implant strength/durability and size provides unique challenges in designing the glenoid half of a total shoulder arthroplasty. Current all-polyethylene glenoid designs have been proven to have inadequate durability to last for the lifetime of the patient.

There have previously been many different designs to try to improve the longevity of glenoid implants; including metal-backed glenoid components, multiple peg configurations, varied keels, and bone-ingrowth polyethylene designs. However, all have either shown no improvement in longevity, or had significant problems including component fracture, early catastrophic wear of the implant, early loosening around the component, and osteolysis (bone destruction) around the component.

There are an increasing number of total shoulder arthroplasty procedures being performed in the United States on a yearly basis. This, coupled with the increasing life span of the population, shows that there continues to be a need for a total shoulder prosthesis with improved longevity.

### SUMMARY

Embodiments use a unique combination of materials to produce a total shoulder arthroplasty prosthesis set with improved strength and durability, and superior osseous-integration characteristics.

### BRIEF DESCRIPTION OF DRAWINGS

Embodiments of the invention are illustrated by way of example and not by way of limitation in the figures of the accompanying drawings in which like references indicate similar elements. It should be noted that references to “an” or “one” embodiment in this disclosure are not necessarily to the same embodiment, and such references mean “at least one.”

FIG. 1 shows a perspective view of a complete prosthesis set according to an embodiment of the invention.

FIG. 2 shows a perspective view of a complete prior-art prosthesis set.

FIGS. 3A and 3B are perspective views of the glenoid component of an embodiment of the invention.

FIG. 4 shows front, back, top, bottom and side views of the glenoid component of an embodiment.

FIGS. 5A and 5B show another glenoid component according to an embodiment of the invention.

FIG. 6 illustrates measurements and features of a glenoid component.

FIG. 7 shows several views of a humeral head prosthesis according to an embodiment.

FIGS. 8 and 9 show views of a humeral stem portion of an embodiment.

FIG. 10 shows another view of a humeral stem portion of an embodiment.

### DETAILED DESCRIPTION

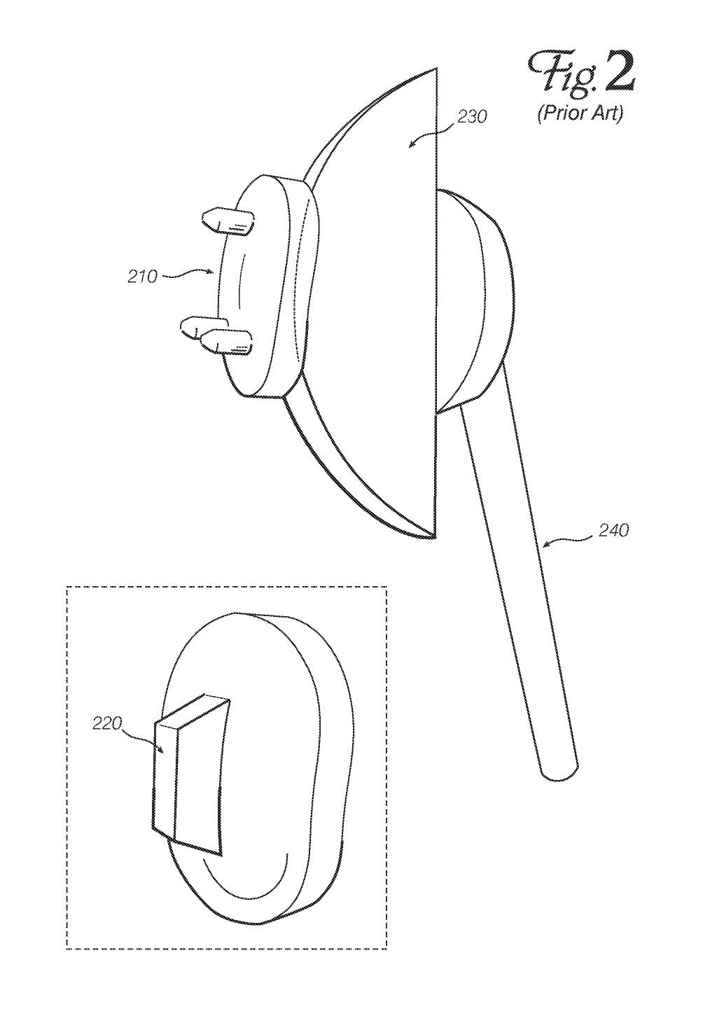

A description of the standard total shoulder prosthesis provides a good starting point to understand the characteristics that make an embodiment of the invention different from the prior art. In the standard prosthesis (referring to FIG. 2), a thin (Ca. 3-4 mm thick) polyethylene (“PET”) cup 210 having cylindrical pegs on its back (bone-contacting, convex) side is implanted onto the scapula and secured there using adhesive in hole(s) corresponding to the pegs. (Some prior-art glenoid prostheses have a polyhedral keel that is cemented into a slot cut into the scapula, as shown at 220 in the inset.) This cup replaces the glenoid side of the joint.

On the humeral side, either a portion of the humeral head or the entire humeral head is replaced by a metal hemisphere 230 (shown partially cut away in this Figure) whose radius is somewhat smaller than or equal to the concave radius of curvature of the face of glenoid cup 210. The metal hemisphere 230 may be secured directly to the humerus, or to a stem 240 inserted into the humerus. The hemisphere may be adjustable for position and/or angle to match the glenoid cup.

The most significant problem with this arrangement is that material suitable for the glenoid cup (typically polyethylene) cannot osseointegrate—the bone of the scapula will not grow into and secure the cup. To secure the cup in position, an adhesive is placed in holes drilled to accommodate the pegs (or the keel), and the cup is pressed into place. The cup material itself is relatively soft and flexible, and under the wide range of stresses experienced in the highly mobile shoulder joint, the cup tends to loosen leading to pain and decreased range of motion of the shoulder. Eventually, the cup may completely wear through or even work its way out of position on the scapula.

To address this problem, a metal-backed PET glenoid component (not shown) has been tested but suffered from several significant problems. Since the space available for any glenoid component is quite limited, the PET articular surface when combined with a metal backing leads to an extremely thin PET lining. Although the metal backing permits osseointegration and improved fixation, the thin layer of PET tends to wear quickly, eventually giving way to metal-on-metal contact between the metal backing of the glenoid component and the replacement humeral head leading to early failure. In addition, due to the thickness of the metal and PET construct, the prosthesis was frequently too large which caused problems with restoring the normal gleno-humeral joint relationships. This led to increased pain and decreased motion in these prosthesis, as well as early failure.

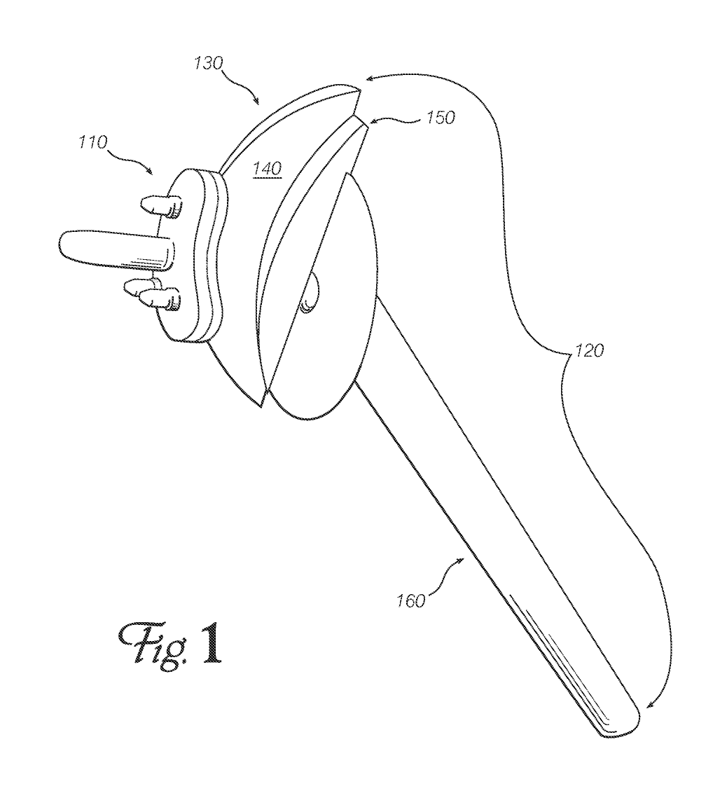

Embodiments of the present invention can be thought of broadly as swapping the materials of the parts of a standard total shoulder prosthesis. In an embodiment (turning to FIG. 1), the entire glenoid cup 110 is a monolithic structure of a biocompatible solid (e.g., titanium, cobalt chrome, stainless steel or ceramic) with a smooth, concave depression (not visible in this view) replacing the glenoid fossa, and a humeral component 120 having a ball head 130 with a softer, slightly compliant articular surface made of PET or a similar substance 140 bonded to a biocompatible solid backing 150, which may be secured in place of the humeral head or to a stem 160 that is inserted into the humerus. Since the humeral prosthesis is physically larger than the glenoid portion, it can be constructed with a thick layer of articular material 140 for extended service life and improved wear characteristics, while yet leaving room to bond the articular material securely to the biocompatible solid base plate 150 that is fixed in place of the humeral head, or secured to stem 160 which is inserted into the bone of the humerus.

“Solid,” “biocompatible solid,” and like terms and phrases in the present disclosure should be understood to mean “bone-like materials” and “materials that are suitable for long-term implantation into bony sites.” Biocompatible solid structures according to embodiments of the invention need not be formed out of monocrystalline bulk materials or milled or cast from material so as to have no included voids. Suitable structures may be milled or cast, but may also be formed, for example, by sintering metal powder. Sintered metal may be fairly porous, and therefore may not meet a strict dictionary definition for “solid,” but it still may be suitable for use in an embodiment.

The important characteristics of a biocompatible solid with respect to embodiments is that they can be installed within the human body without excessive risk of rejection or other adverse reactions; that they be similar in strength and flexibility to the bone in which they are implanted; and that they are (or can be treated to be capable of) osseointegration (becoming securely fused to the host bone via ingrowth or similar processes). Portions of a biocompatible solid structure may receive special treatment to produce particular characteristics over portions of their surface or volume. For example, some surfaces may be heat-treated, mechanically impacted, irradiated, polished or plated, or a surface may be coated or treated with a substance to promote bone ingrowth. By way of contrast, the PET used for prior-art glenoid cups might satisfy some characteristics of a biocompatible solid, but it is not similar in strength and flexibility to bone (of similar volume/dimension), and it cannot osseointegrate. Therefore, a prior art PET glenoid is not a “biocompatible solid glenoid prosthesis” within the meaning of those words in this disclosure.

Embodiments of the invention include several components:

### Solid Glenoid Component

The glenoid prosthesis is preferably a single-piece, solid structure formed of a biocompatible solid. It has a bone-interfacing side (FIG. 3A) with a plurality of structures suited for securing the prosthesis to a patient's scapula, and a concave articular-surface side (FIG. 3B) opposite the bone-interfacing side. The securing structures of the bone-interfacing side may be cylindrical pegs that are inserted into holes drilled in the scapula. The articular surface has a smooth and preferably polished finish.

The inherent strength of having a solid (e.g., all metal) component allows the glenoid component to be thin compared to a poly or metal-poly hybrid component. Having a thinner component prevents lateralization of the joint (overstuffing) which leads to poor outcomes in total shoulder arthroplasty, and is a significant problem with metal-polyethylene hybrid glenoid components.

A solid (e.g., all metal) component allows for direct bone ingrowth into back of the glenoid component itself, creating a permanent bond between the implant and the bone to allow for improved long term stability and retention of the implant.

No modularity of the glenoid component. The highly polished face and body are formed or directly bonded together. Decreasing the modularity of the component allows for fewer areas of the component which could be susceptible to fatigue stress and failure. As there is preferably no PET on this half of the component, this removes the possibility of backside polyethylene wear which was a significant problem with metal-polyethylene hybrid glenoid components.

Flanges on the peripheral pegs of the implant prevent cement extrusion onto the bony ingrowth surface of the glenoid component when the implant is placed using cement.

Implant placed within the glenoid itself instead of on top of the glenoid bone, which allows for osseointegraion of the both the pegs as well as the body of the component itself.

Bone ingrowth coating or surface treatment on back of the implant itself in addition to the pegs which protrude for the back of the implant, allowing for a greater surface area for bony fixation, and improved resistance to loosening.

Humeral head:

The humeral head of an embodiment replaces the patient's humeral head with a substantially hemispherical structure having an outer (glenoid-facing) surface of a tough, resilient, slightly flexible material. The material should be able to slide, turn and rotate (generally, “articulate”) smoothly against the concave glenoid articular surface. This material may be formed and bonded onto a solid, substantially planar mounting surface opposite the convex hemisphere. The planar mounting surface may be made of the same biocompatible solid as the glenoid component, or a different biocompatible solid. The mounting surface may be suitable for securing directly to the patient's humeral bone, to a complementary mounting surface attached to the bone, or to a complementary mounting surface of a stem that is inserted into the patient's humerus. In any of these arrangements, the convex, glenoid-facing ball is oriented and aligned to fit into the concave depression of the glenoid prosthesis.

The convex hemisphere may be made of moderately or highly cross-linked ultra high molecular weight (UHMW) polyethylene. This material allows for a significant reduction in wear rates compared to standard UHMW polyethylene.

With a UHMW humeral head (as opposed to a UHMW glenoid), the polyethylene can be much thicker (2-3× or more). Thicker polyethylene has significantly improved wear characteristics compared to thin polyethylene which again allows for a decrease in wear of the implant.

Direct bonding of the polyethylene to a metal mounting surface or base plate prevents any backside wear of the polyethylene head.

### TECHNICAL DETAILS

### Glenoid Component

### Glenoid Component Body

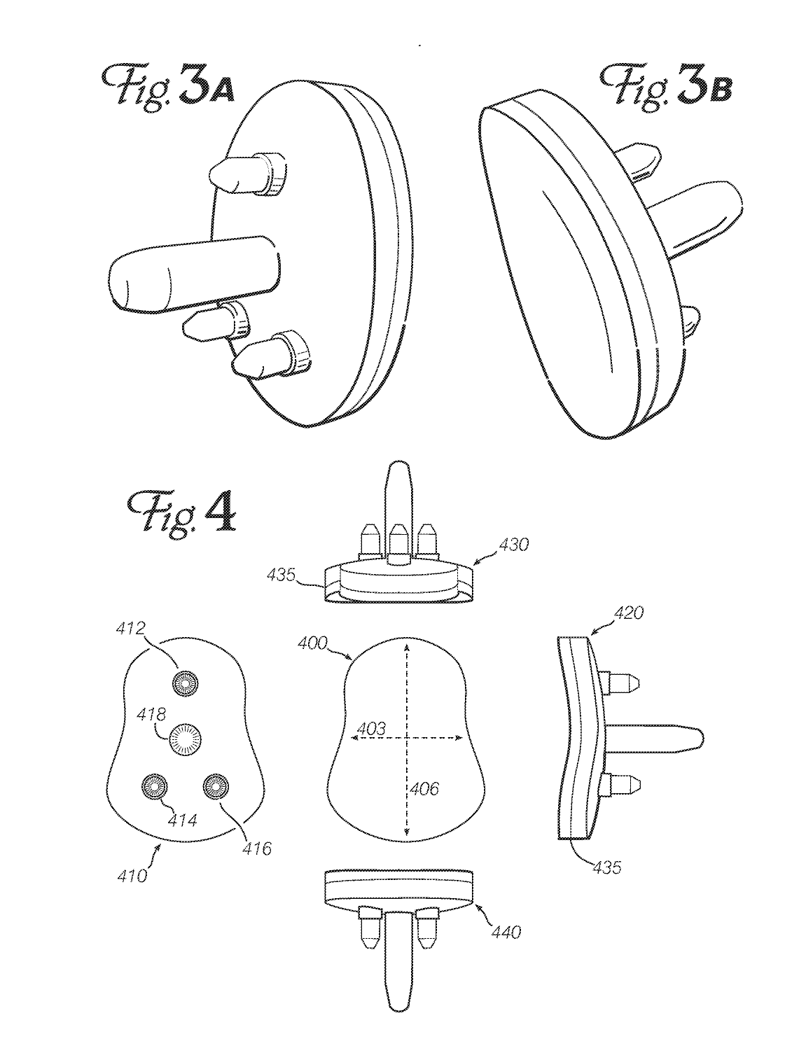

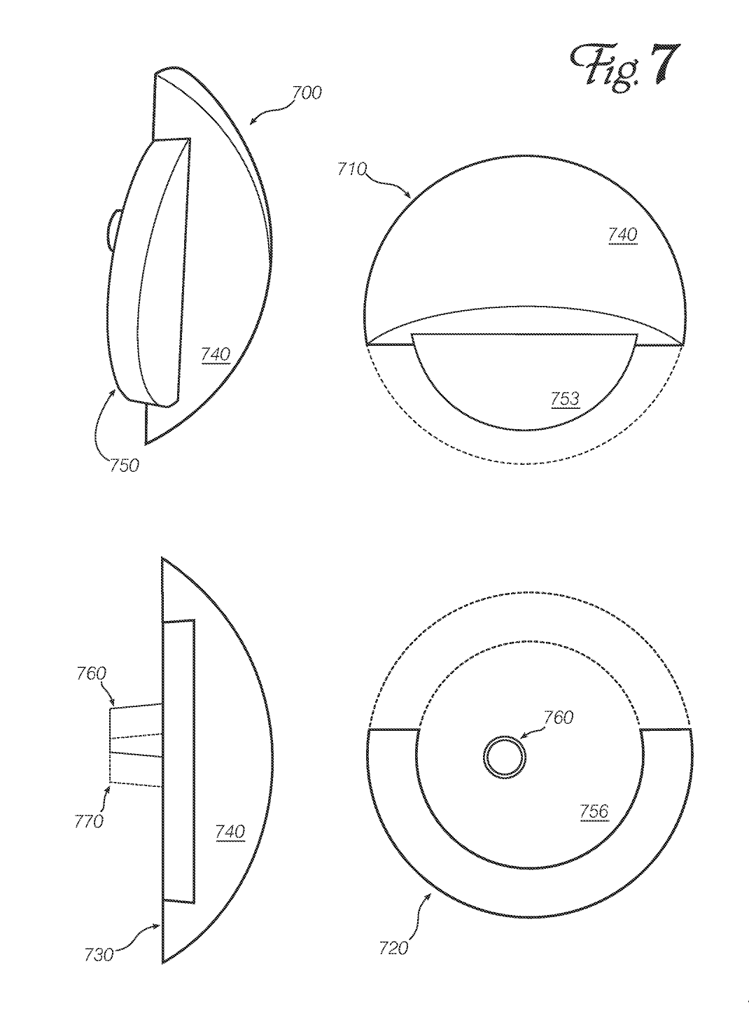

The component body (referring generally to FIGS. 3A, 3B, 4 and 5) may be made of cobalt chrome, titanium, ceramic or other suitable biocompatible metal. This serves as the backing for the articular face of the component and allows for initial stable fixation of the glenoid into the host bone, and also for osseous integration of the component into the host bone. The bone side of the component body (the side in direct contact with the host bone, including the sides which are embedded or recessed into the bone) may be coated in a porous ingrowth surface such as sintered metal beads, hydroxyapatite, plasma-spray, or porous tantalum to allow ingrowth of the host bone directly into the back of the body of the component or may be made directly out of an ingrowth surface such as porous tantalum or ceramic. In these Figures, a broken line seen on the side of the glenoid face (e.g., FIGS. 4, 425 and 435) indicates a boundary line between the ingrowth-coated backside and the non-coated, polished articular face. Placing porous coating on the entire back of the body of the component is important in that when fully grown-in it prevents additional shear force across any pegs on the back of the component. There may be several smaller additional pegs (e.g., three additional pegs, as shown in the Figures, and identified in the back side orthogonal view, FIGS. 4, 410, at 412, 414 and 416) protruding from the bone side of the body of the component. These may be about 3-5 mm long by about 1-3 mm in diameter. There may be one on the superior aspect of the body (412) which is directly midline, and two which may be on the inferior aspect (414, 416) and which may be offset about 2-4 mm from the vertical midline of the component. The smaller pegs may be coated in a porous ingrowth surface or may be smooth with small (less than 0.5 mm) grooves cut in them. The smooth version of the smaller peg is intended for cement fixation. In this embodiment, the pegs may have a small, approximately 1 mm collar near the back surface of the component (refer to FIG. 6, 640) to prevent cement extrusion onto the ingrowth backing of the glenoid when being implanted with cement. A large central peg (FIG. 4, 418) in the center of the bone side of the component should also be present, approximately equidistant from any peripheral small pegs. The large peg may have an approximate diameter of 5-8 mm and an approximate length of 5-15 mm. This large central peg may be fully coated in a porous ingrowth surface to facilitate stable bony ingrowth. The glenoid component body is preferably implanted (embedded) within the glenoid bone itself for a depth of approximately 1-2 mm (refer, for example, to FIG. 6: the back portion 660 is embedded into the glenoid bone, while the front portion 665 may extend beyond the bone surface). This is beneficial in that it allows the implant body to fully osseointegrate with the bone of the glenoid and to be better supported.

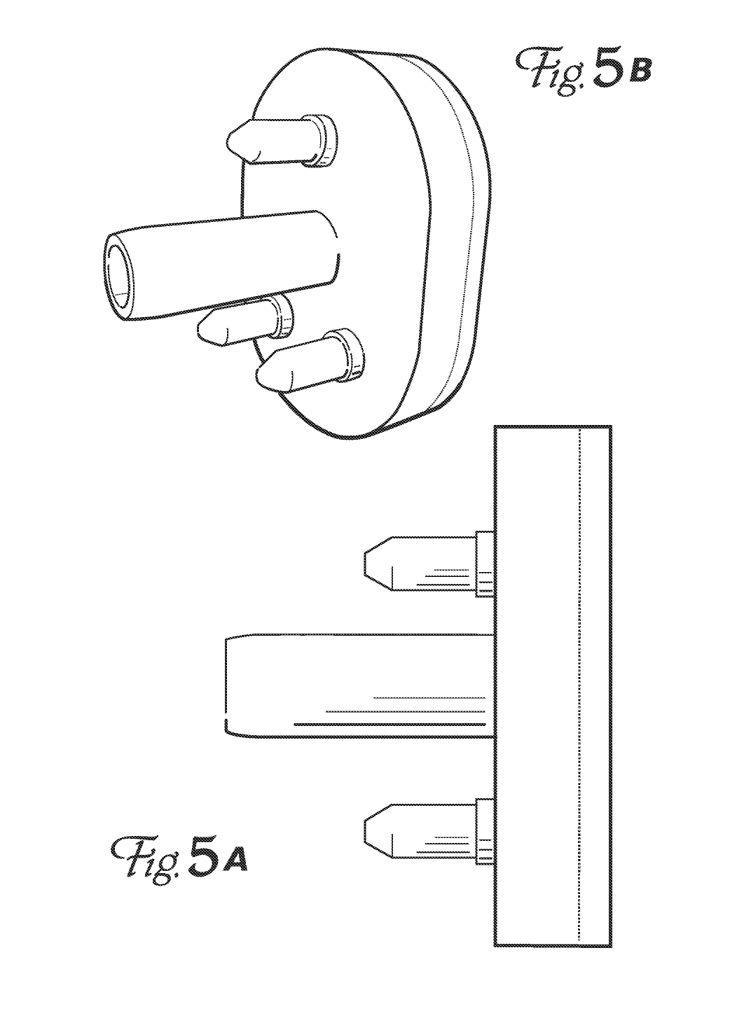

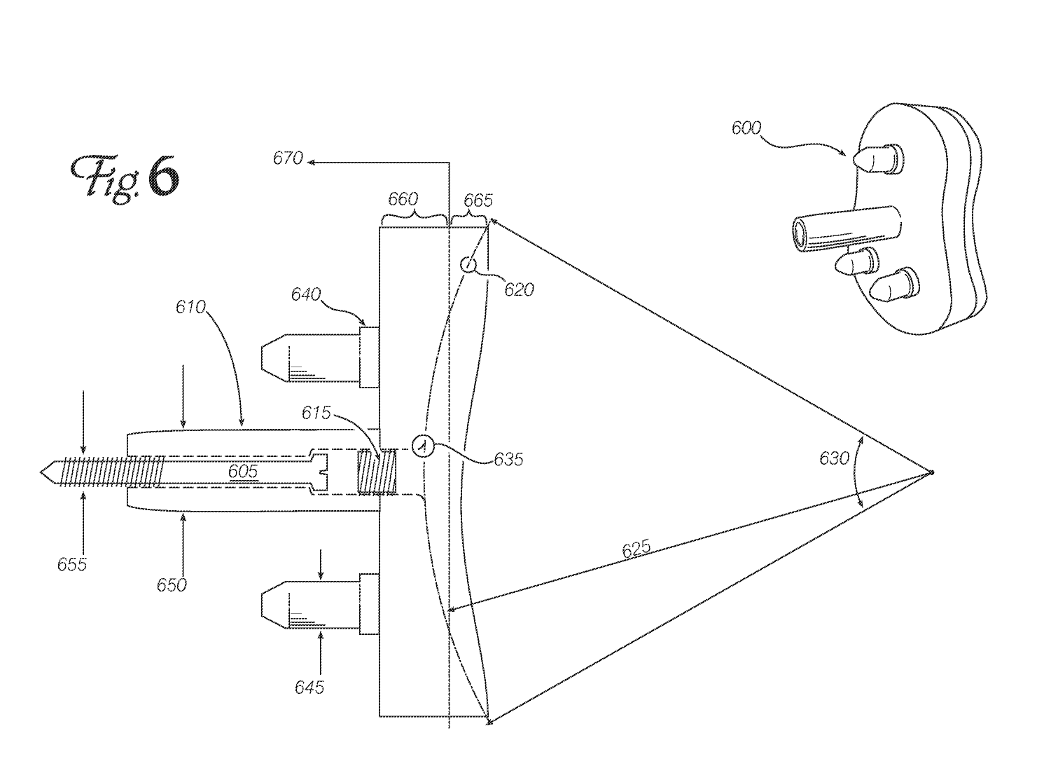

The back or bone-interfacing side of the glenoid component may be convex (FIGS. 1, 3A, 3B, 4) or flat (FIGS. 5A, 5B, 6). The curvature of the back side (if any) may be similar to the curvature of the articular surface, or different therefrom.

### Glenoid Articular Face

The face of the glenoid component may be made of highly polished cobalt chrome, hardened titanium, ceramic, or other wear resistant metal (refer to FIG. 3B and front orthogonal view 400 in FIG. 4). The thickness of the articular surface may be about 0.5-2 mm. The diameter of the face of the component may range from about 40 mm to about 58 mm. The radius of curvature is preferably near that of the corresponding humeral head but may allow for 1-10 millimeters of mismatch as needed. The articular face is bonded directly to underlying glenoid body component. The smooth surface should sit just above the bony surface of the glenoid to prevent excessive lateralization of the glenoid component known as “over-stuffing the joint.”

The glenoid face “diameter” measurement mentioned above implies a circular glenoid face profile. However, as shown in these figures, the actual profile may take other shapes. For example, FIGS. 3A and 3B show an elliptical or oval glenoid; and FIG. 4 shows a pear or saddle-shaped glenoid (400). The face may also be circular (not shown) or a round-ended oblong (e.g. FIG. 5A). For shapes that do not have a clearly defined “diameter,” one may usefully specify the glenoid size in terms of its articular surface area. Most embodiments will have an area roughly similar to that of a natural glenoid, or about 4-10 cm².

### Humeral Component:

Humeral stem:

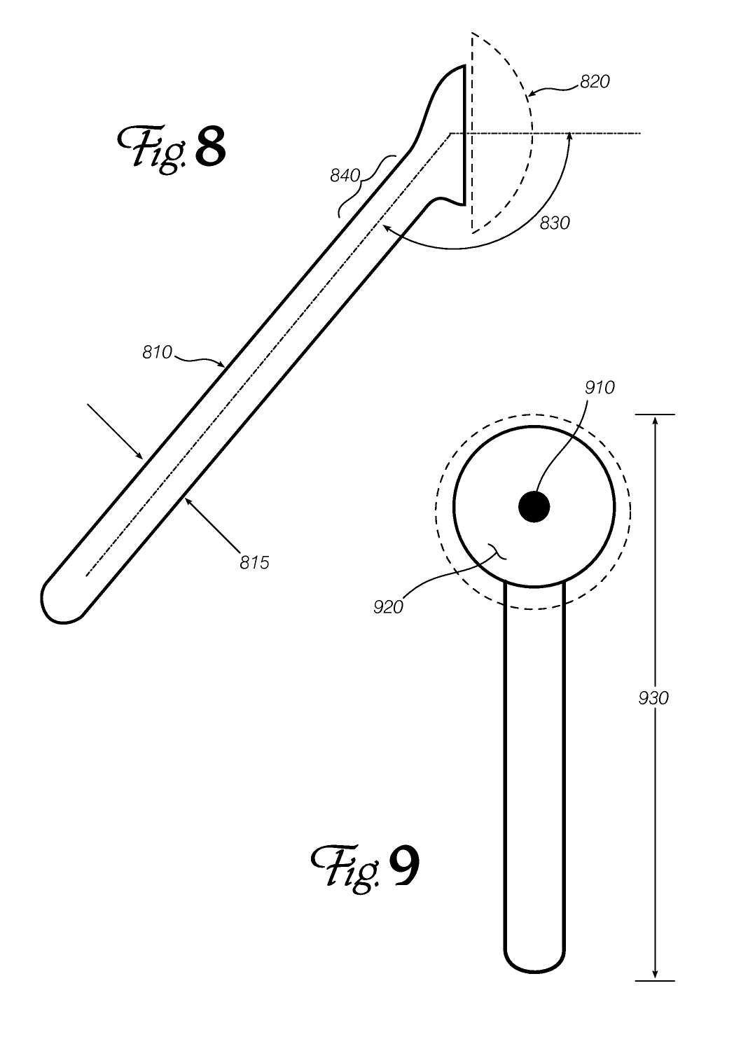

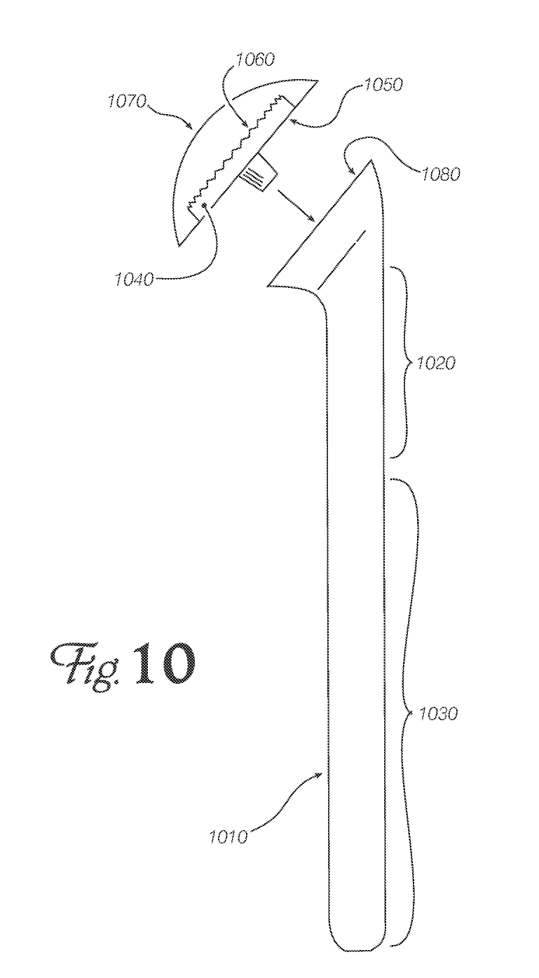

The humeral stem component may be made of a standard biocompatible metal such as titanium or cobalt chrome. The neck angle of the implant should be between about 130 and about 145 degrees (see FIG. 8, 830). The diameter of the straight “rod” portion of the humeral stem may range from about 4 mm to about 18 mm. The length of the implant from superior to most inferior is preferably between 120 mm and 180 mm (FIG. 9, 930), and components should be available in different lengths to allow for varying patient anatomy. The rod of the humeral stem is inserted into the medullary cavity or canal, a roughly cylindrical tube extending much of the length of the humerus. The metaphyseal (proximal) aspect of the humeral implant (FIG. 8, 830) preferably widens to fill the metaphyseal portion of the humerus. The mounting face of the most proximal end (FIG. 9, 920) will preferably be flat, circular, and highly polished. In the center of the most proximal end will be a receiving (female) end of a Morse taper (910) (or similar) locking mechanism with a diameter of approximately 4-8 mm. The metaphyseal region of the implant (FIG. 10, 1020) will preferably be coated or treated with a porous ingrowth surface to allow for bony ingrowth. This may extend from the most proximal end of the implant down the stem for a length of 15-40 mm. In a preferred embodiment, the portion of the humeral stem distal to this (1030) may be polished smooth.

### Humeral Head

In preferred embodiments, the humeral head component is made of two distinct materials bonded together to make a single component. (Refer principally to FIG. 7, showing a perspective view 700 and front, back and side views 710, 720, 730. Views 700-720 are cut away to show internal structure.) The articular portion of the component (740, the convex portion that articulates with the glenoid component) may be made of highly or moderately crosslinked UHMW polyethylene. This component may be manufactured in various backside diameters from about 40 mm to about 58 mm, and in various thicknesses and radii of curvature to accommodate varying anatomy among patients. The back side of the humeral head, the base plate 750, has two faces: one that is bonded directly into the UHMW polyethylene articular portion, and one that has the corresponding male portion of the Morse taper 760 (or other locking mechanism) which is impacted into the humeral stem. The base plate should be about equal in diameter to the most proximal, circular portion of the humeral stem. The side of the base plate that bonds to the polyethylene may have a rough or figured surface (see FIG. 10, 1060) to allow for bonding and interlocking with the polyethylene head during production of the implant. The face that is impacted into the humeral stem has a male portion or “shank” of the Morse taper corresponding to the female or “socket” Morse taper on the humeral stem. The humeral-head Morse taper 760 may be located eccentrically (displaced slightly from the center of the circular base plate). An eccentric location of the humeral head Morse taper allows for adjustment by rotation of the humeral head to fully cover any exposed humeral bone. However, centrally-located Morse tapers (e.g., 770) may also be used in embodiments requiring less adjustment. The back side base plate 750 should be flush with the polyethylene head 740 with the exception of the male portion of the Morse taper. It is appreciated that the male and female portions of the interlocking Morse taper may be reversed as between the humeral head and the humeral stem (i.e. , the humeral head may have a female Morse taper, while the most proximal end of the stem may have the corresponding male protrusion). The base plate need not be capable of osseointegration, since it is joined to the humeral stem via a solid-to-solid connection such as interlocking Morse tapers. Thus, the material of the base plate may be different from the material of the glenoid prosthesis and/or of the humeral stem.

### IMPLANTATION TECHNIQUE

### Glenoid Component

This implant is intended to be placed within vault of the glenoid by any combination of a hybrid cement-press fit technique, a solely press-fit fixation, by one or more screws, or by a combination of such techniques. With hybrid . cement-press fixation, the glenoid face is reamed clown to a depth to allow 0.5-2 mm of countersinking of the implant into the glenoid bone. Three small peripheral holes are drilled into the bone, corresponding to the diameter and location of the peripheral pegs of the implant. A larger central hole is drilled which corresponds to the central peg on the glenoid implant, but is slightly smaller in diameter to allow for excellent press-fit fixation. The small peripheral peg holes may be filled with polymethyl-methacrylate (PMMA) cement and the implant is impacted into the glenoid face and held until the PMMA cement is fully hard. (Flanges on the peripheral peg holes, e.g. at FIG. 6, 640, help prevent the PMMA cement from escaping from the peripheral holes and coating parts of the back of the implant. Cement coating may interfere with bone ingrowth, so the flanges are helpful to achieve better long-term implant fixation.) With only press-fit fixation, the peripheral holes may also be drilled to a slightly smaller diameter than the pegs themselves to allow for secure press-fit fixation. The remaining steps are the same.

In an alternate embodiment, referring principally to FIG. 6 (general perspective representation at 600), the central peg is hollow, and a threaded fastener 605 inserted through the central peg 610 can help hold the glenoid component in place until bone ingrowth occurs. In such an embodiment, a portion of the hollow channel near the articular surface may be threaded to accept a sealing plug 615. This plug both prevents back out of the threaded fastener, and separates the back or bone-interfacing side of the component from the front or articular-surface side, preventing fluid from the joint area from passing through the hollow central peg to the bone side, where it might cause osteolysis and early loosening.

FIG. 6 also illustrates a number of measurements that help define an embodiment of the invention. Broken line 620 shows a section of the concave glenoid articular surface, whose radius 625 is preferably between 37 mm and 65 mm (different radii are suitable for patients of different sizes). The angle 630 subtended from one extreme of the glenoid articular surface to another extreme thereof is between about 26° and about 95° (geometrically speaking—the minimum is with a small-diameter or small-surface-area glenoid and a large radius of curvature, while the maximum is with a large glenoid and small radius). Practically speaking, the angle subtended is more likely to range from about 40° to about 55°.

In an embodiment with a hollow main fixing structure suited to accept a threaded fastener, the edge of the hole in the articular surface should be rounded or beveled as shown at 635 to prevent wear of the humeral head as it moves against the articular surface.

As previously described, peripheral pegs may have a diameter 645 of about 1-3 mm and a length of about 3-5 mm. The main fixation structure (peg) may have a diameter 650 of about 5-8 mm (even larger diameters may be necessary to accommodate a threaded fastener 605 of preferred diameter 655 of about 6.5 mm).

FIG. 6 also indicates how the glenoid component may be partially embedded in the glenoid bone: the back portion including the securing structures and the sides to a depth 660 (perhaps 0.5-1.5 mm) are countersunk into the bone (bone surface may be near 670) while the outer portion of the glenoid component 665 may stand proud of the bone by a small amount.

### Humeral Component

This implant is intended to be placed in the humeral shaft and metaphyseal region of the humerus. It may be installed with a press-fit only technique or a cemented technique. The patient's humeral head is removed at the appropriate resection angle and depth. The metaphyseal bone is reamed/compacted to an appropriate amount to allow passage of the humeral stem (e.g., FIG. 8, 810). The stem is then placed in the depth and rotation as decided by the surgeon, entering the medullary cavity or canal of the humerus. A humeral head 820 corresponding to the glenoid curvature, allowing for mismatch, would then be chosen. Using trial components, the proper rotation, thickness, and diameter of the humeral head would be determined. Then a humeral head component 820, as detailed in FIG. 7, corresponding to the selected trial size would be placed on the superior face of the stem (FIG. 9, 910), and the two ends of the Morse taper (920 and e.g. FIG. 7, 760) joined together. Using an impact device, the Morse taper would be locked together in the appropriate rotation.

### Arthroplasty Kit

Embodiments of the invention may be supplied as kits containing a plurality of glenoid prostheses as described above, in varying sizes (e.g., different-sized securing structures [pegs], different face diameters or surface areas, and different radii of curvature), and another plurality of humeral head prostheses of the structure described above (tough, resilient hemisphere bonded to mounting plate), where the heads also vary in backside diameter and convex radii of curvature. One or more humeral stems may also be provided to suit different humerus sizes (diameters and lengths). The mounting face and features of the humeral stems should match those of the humeral heads. The kit may also include trial humeral heads of varying sizes, which can be temporarily joined to an implanted humeral stem to test for smooth action against the implanted glenoid prosthesis. When a suitable trial head is located, it can be replaced with a permanent head of the same size from the kit.

The present invention has been described largely by reference to specific examples and in terms of combinations of features in an embodiment. However, those of skill in the art will recognize that glenohumeral prostheses according to an embodiment of the invention can have different sizes, shapes and arrangements without departing from the inventive principles disclosed herein. Such variations are understood to be captured according to the following claims.

1. A prosthesis set for replacing a human shoulder joint comprising a glenoid prosthesis and a humeral prosthesis, the glenoid prosthesis comprising:
a monolithic biocompatible solid structure having a bone-interfacing side oriented in a first direction and a concave glenoid depression side oriented in a second direction opposite the first direction, a surface joining an outer perimeter of the bone-interfacing side to an outer perimeter of the concave glenoid depression side forming a glenoid cup side for at least partial embedding in a glenoid bone of a patient,
the concave glenoid depression side having a smooth glenoid surface, and
the bone interfacing side including at least one securing structure,
the bone interfacing side, the at least one securing structure and at least part of the glenoid cup side having an ingrowth surface to promote osseous integration between the the ingrowth surface and a scapula of the patient;
and the humeral prosthesis comprising:
a humeral-head replacement having a convex glenoid-articulating face oriented in a third direction and a humeral head mounting face oriented in a fourth direction opposite the third direction,
the convex glenoid-articulating face formed of a tough material that articulates smoothly against the glenoid surface,
wherein the glenoid prosthesis and the humeral prosthesis are related as:
a glenoid radius of curvature of the concave glenoid depression side is no smaller than a humeral head radius of curvature of the convex glenoid-articulating face.

2. The prosthesis set of claim 1 wherein the humeral prosthesis further comprises:
a biocompatible solid humeral stem having a rod portion coupled to a stem mounting face through an angled neck portion, the stem mounting face configured for substantially permanent fixation to the humeral head mounting face and the rod portion suitable for inserting into a medullary cavity of a humerus of a patient.

3. The prosthesis set of claim 2 wherein the stem mounting face comprises a first interlocking feature and the humeral head mounting face comprises a second, complementary interlocking feature.

4. The prosthesis set of claim 3 wherein one of the first and second interlocking features is a Morse taper shank and another of the first and second interlocking features is a corresponding Morse taper socket.

5. (canceled)

6. The prosthesis set of claim 1 wherein the ingrowth surface includes a coating selected from the group consisting of a hydroxyapatite coating, a sintered metal bead coating, a plasma spray coating, a ceramic coating, and a porous tantalum coating.

7. The prosthesis set of claim 1 wherein the at least one securing structure includes a cylindrical peg.

8. The prosthesis set of claim 7 wherein the cylindrical peg comprises a hollow channel to allow a threaded fastener to pass from the concave glenoid depression side through to the bone-interfacing side.

9. The prosthesis set of claim 8 wherein a portion of the hollow channel is threaded, the prosthesis set further comprising:
a threaded sealing plug to match the threaded portion of the hollow channel, the threaded sealing plug operative to perform at least one of:
preventing the threaded fastener from backing out; and
preventing fluid from passing through the hollow channel between the concave glenoid depression side and the bone-interfacing side of the glenoid prosthesis.

10. The prosthesis set of claim 1 wherein the at least one securing structure includes a larger central peg located roughly equidistantly from a plurality of smaller peripheral pegs.

11. The prosthesis set of claim 10 wherein the plurality of smaller peripheral pegs have a smooth, non-ingrowth surface.

12. The prosthesis set of claim 11 wherein the plurality of smaller peripheral pegs each have a flange near a glenoid end of the peripheral peg.

13. The prosthesis set of claim 1 wherein the concave glenoid depression subtends an angle about its center of curvature and from one extreme edge to another extreme edge, said angle between about 40° and about 55°.

14. The prosthesis set of claim 1 wherein a front orthogonal view of the concave glenoid depression is one of a circle, an oval, a round-ended rectangle, or a pear shape.

15. The prosthesis set of claim 1 wherein a diameter of the concave glenoid depression is between about 40 mm and about 58 mm.

16. The prosthesis set of claim 1 wherein the humeral head radius of curvature is between about 1 mm less than the glenoid radius of curvature and about 10 mm less than the glenoid radius of curvature.

17. The prosthesis set of claim 1 wherein the convex glenoid-articulating face is ultra high molecular weight (“UHMW”) polyethylene.

18. The prosthesis set of claim 1 wherein the convex glenoid-articulating face is crosslinked UHMW polyethylene.

19. A prosthesis set for replacing a human shoulder joint, comprising a glenoid replacement prosthesis and a humeral head replacement prosthesis, the glenoid replacement prosthesis comprising:
a unitary biocompatible solid body formed principally of a material selected from the group consisting of titanium, cobalt chrome, stainless steel and ceramic, said solid body having a bone-interfacing side oriented in a first direction and a concave glenoid face oriented in a second direction opposite the first direction,
the bone-interfacing side having a plurality of securing pegs and an ingrowth surface covering the bone-interfacing side, at least one of the securing pegs and at least a portion of an embeddable glenoid-cup side surface,
the concave glenoid face having a polished surface with a surface area between about 4 cm² and about 10 cm²,
the concave glenoid face having a radius of curvature between about 37 mm and about 65 mm; and
the humeral head replacement prosthesis comprising:
a convex humeral head component with a crosslinked ultra-high molecular weight (“UHMW”) polyethylene ball portion bonded to a mounting plate portion, said mounting plate portion having a substantially permanent interlocking feature and
a radius of curvature of the convex humeral head component being smaller than the radius of curvature of the concave glenoid face;
a humeral stem component with a rod portion joined to a mounting face through an angled neck portion,
said rod portion configured to enter a medullary canal of a humerus of a patient,
a proximal portion of said rod portion enlarged to fill a corresponding metaphyseal portion of the humerus and having a bone ingrowth surface thereupon, and
the mounting face having an interlocking feature complementary to the substantially permanent interlocking feature of the mounting plate.

20. A prosthesis kit for replacing a human shoulder joint, the kit comprising:
a plurality of solid biocompatible glenoid prostheses, each such glenoid prosthesis composed mainly of one of titanium, cobalt-chrome, stainless steel or ceramic, and having:
a bone-interfacing side with a plurality of cylindrical pegs, the bone interfacing side, at least one of the cylindrical pegs and at least a portion of an embeddable glenoid-cup side surface having a porous bone-ingrowth surface; and
a concave glenoid depression side with a polished surface,
said plurality of solid biocompatible glenoid prostheses differing in a surface area of their glenoid depression sides and in a radius of curvature of their concave depressions;
a plurality of humeral head prostheses, each such humeral head prosthesis comprising a convex ultra-high molecular weight (“UHMW”) crosslinked polyethylene hemispherical portion bonded to a backing plate with a semi-permanent interlocking feature,
said plurality of humeral head prostheses differing in backside diameter and radii of curvature of the convex hemispherical portion; and
a humeral stem component composed mainly of one of titanium, cobalt-chrome or stainless steel, said humeral stem component having
a rod suited for insertion into a humeral medullar cavity of a patient,
a circular mounting face having an interlocking feature complementary to the semi-permanent interlocking feature of the plurality of humeral head prostheses, and
an angled neck coupling the rod to the circular mounting face.
